(19) United States
(12) Patent Application Publication
(10) Pub. No.: US 2023/0037465 A1
(43) Pub. Date: Feb. 9, 2023

(54) SYSTEMS AND METHODS FOR ALTERNATE MODES IN AUTOMATED INSULIN DELIVERY FOR DIABETES THERAPY

(71) Applicant: Tandem Diabetes Care, Inc., San Diego, CA (US)

(72) Inventors: Jose Ricardo Rueda, LaJolla, CA (US); Nicholas Sherer, Escondido, CA (US); Thomas R. Ulrich, Oceanside, CA (US); Katherine Vyvy Tran, San Diego, CA (US); Pat Mulvihill, San Diego, CA (US); Peter Zhao, San Diego, CA (US); Micah Stephens, San Diego, CA (US); Ryan Cardenas, San Diego, CA (US); Paul Harris, San Diego, CA (US); Geoffrey A. Kruse, San Diego, CA (US); Michael Michaud, San Diego, CA (US); Virginia S. Lu, San Diego, CA (US)

(21) Appl. No.: 17/732,208

(22) Filed: Apr. 28, 2022

Related U.S. Application Data
(60) Provisional application No. 63/228,884, filed on Aug. 3, 2021.

(51) Int. Cl. A61M 5/172; G16H 40/63; G16H 20/17

(52) U.S. Cl. CPC A61M 5/1723; G16H 40/63; G16H 20/17; A61M 2205/502; A61M 2230/201; A61M 2205/18

(57) ABSTRACT

Disclosed herein are systems and methods for automated insulin delivery that provide an Alternate Normal Activity Mode that has a lower and narrower target range than the standard range employed in Normal Mode. The Alternative Normal Mode can be a user-selectable feature that provides more aggressive glucose level control that is designed to decrease hyperglycemia without significantly increasing the risk of hypoglycemia. For example, Normal Mode may employ a standard glucose range between which the closed loop algorithm attempts to maintain the user's glucose levels such as 112.5 mg/dL to 160 mg/dL and Alternative Normal Mode may employ a lower and narrower range such as 90 mg/dL to 130 mg/dL. Alternative Normal Mode can also employ a lockout feature that is activated when glucose is high, but is falling rapidly.

### RELATED APPLICATION

This application claims the benefit of U.S. Provisional Application No. 63/228,884 filed Aug. 3, 2021, which is hereby fully incorporated herein by reference.

### FIELD OF THE INVENTION

The present invention relates generally to ambulatory infusion pumps and, more particularly, to operation of ambulatory infusion pumps in a closed-loop or semi-closed-loop fashion.

### BACKGROUND OF THE INVENTION

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices that can provide continuous subcutaneous insulin injection and/or infusion therapy as an alternative to multiple daily insulin injections via syringe or injector pen. Such ambulatory infusion pumps may be worn by the user, may use replaceable medicament cartridges, and may deliver other medicaments alone, or in combination with insulin. Such medicaments include glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as continuous glucose monitoring (CGM) devices. A CGM device consists of a sensor placed under the patient's skin and affixed to the patient via an adhesive patch, a transmitter, and a monitor. A CGM device samples the patient's interstitial fluid periodically (e.g. once every 1-5 minutes) to estimate blood glucose levels over time. CGMs are advantageous because they provide more frequent insights into a user's blood glucose levels yet do not require a finger stick each time a reading is taken.

Ambulatory infusion pumps may incorporate a CGM within the hardware of the pump or may communicate with a dedicated CGM directly via a wired connection or indirectly via a wireless connection using wireless data communication protocols to communicate with a separate device (e.g., a dedicated remote device or a smartphone). One example of integration of ambulatory infusion pumps with CGM devices is described in U.S. Patent Publication No. 2014/0276419, which is hereby incorporated by reference herein. Ambulatory infusion pumps typically allow the user or caregiver to adjust the amount of insulin or other medicament delivered by a basal rate or a bolus, based on blood glucose data obtained by a CGM device, and in some cases include the capability to automatically adjust such medicament delivery. For example, based on CGM readings, some ambulatory infusion pumps may automatically adjust or prompt the user to adjust the level of medicament being administered or planned for administration or, in cases of abnormally low blood glucose readings, reducing or temporarily ceasing insulin administration.

In some cases, ambulatory insulin pumps may be configured to deliver insulin based on CGM data in a closed-loop or semi-closed-loop fashion. Some systems including these features may be referred to as automated insulin delivery (AID) systems or artificial pancreas systems because these systems serve to mimic biological functions of the pancreas for persons with diabetes.

### SUMMARY

Disclosed herein are systems and methods for automated insulin delivery that provide an Alternate Normal Activity Mode that has a lower and narrower target range than the standard range employed in Normal Mode. The Alternative Normal Mode can be a user-selectable feature that provides more aggressive glucose level control that is designed to decrease hyperglycemia without significantly increasing the risk of hypoglycemia. For example, Normal Mode may employ a standard glucose range between which the closed loop algorithm attempts to maintain the user's glucose levels such as 112.5 mg/dL to 160 mg/dL and Alternative Normal Mode may employ a lower and narrower range such as 90 mg/dL to 130 mg/dL. Alternative Normal Mode can also employ a lockout feature that is activated when glucose is high, but is falling rapidly to reduce the risk of hypoglycemia from the lower control range.

In an embodiment, an ambulatory infusion pump system can include a pump mechanism configured to facilitate delivery of insulin to a user, a user interface, a communications interface adapted to receive glucose levels from a continuous glucose monitor and at least one processor. The at least one processor can be configured to present on the user interface at least two options for calculating and delivering insulin doses according to a closed loop delivery algorithm during normal activity of the user. The at least two options include a normal activity mode having a first glucose range between which the closed loop delivery algorithm attempts to maintain the user's glucose levels and an alternate normal activity mode having a second glucose range. The processor can receive input through the user interface selecting either the normal mode or the alternative normal activity mode, automatically calculate insulin doses with a closed loop delivery algorithm based on glucose levels received from the continuous glucose monitor according to the selected mode, and automatically deliver the insulin doses calculated by the closed loop delivery algorithm to the user with the pump mechanism. If the user has selected the alternate normal activity mode, a bolus lockout feature can further be activated that selectively prevents delivery of automatic correction boluses that would otherwise be delivered to the user's glucose level being over a high threshold.

In an embodiment, an ambulatory infusion pump system an include a pump mechanism configured to facilitate delivery of insulin to a user, a user interface, a communications interface adapted to receive glucose levels from a continuous glucose monitor and at least one processor. The at least one processor can be configured to present on the user interface at least two options for calculating and delivering insulin doses according to a closed loop delivery algorithm during normal activity of the user. The at least two options include a normal activity mode having a first glucose range between which the closed loop delivery algorithm attempts to maintain the user's glucose levels and an alternate normal activity mode having a second glucose range that is lower and narrower than the first glucose range. The processor can receive input through the user interface selecting either the normal mode or the alternative normal activity mode, automatically calculate insulin doses with a closed loop delivery algorithm based on glucose levels received from the continuous glucose monitor according to the selected mode, and automatically deliver the insulin doses calculated by the closed loop delivery algorithm to the user with the pump mechanism.

### BRIEF DESCRIPTION OF THE DRAWINGS

The invention may be more completely understood in consideration of the following detailed description of various embodiments of the invention in connection with the accompanying drawings, in which:

FIG. 1 is an embodiment of an ambulatory infusion pump for use with embodiments of the disclosure.

FIG. 2 is a block diagram of the ambulatory infusion pump of FIG. 1.

FIGS. 3A-3B are an alternate embodiment of an ambulatory infusion pump for use with embodiments of the disclosure.

FIG. 4 is an embodiment of a CGM for use with embodiments of the disclosure.

FIG. 5 is a schematic representation of a closed-loop insulin delivery algorithm according to the disclosure.

FIG. 6 is a schematic representation of an automatic bolus feature of a closed-loop delivery algorithm according to the disclosure.

FIG. 7 is a schematic representation of alternative delivery modes for a closed-loop delivery algorithm according to the disclosure.

FIG. 8 depicts an exemplary embodiment of a series of user interface screens for selecting alternative delivery modes for closed-loop diabetes therapy according to an embodiment.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

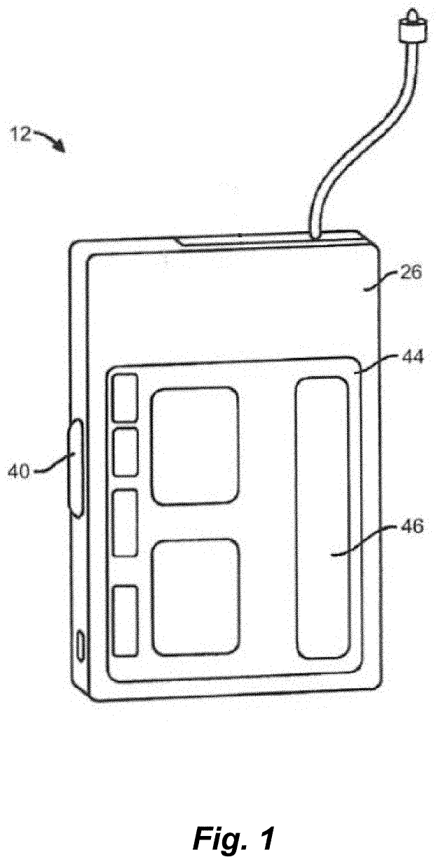

FIG. 1 depicts an example infusion pump that can be used in conjunction with one or more embodiments of the ambulatory infusion pump system of the present disclosure. Pump 12 includes a pumping or delivery mechanism and reservoir for delivering insulin or other medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display (e.g., a dedicated remote display or a CGM display), a remote control device, or a consumer electronic device (e.g., laptop computer, personal computer, tablet computer, smartphone, electronic watch, electronic health or fitness monitor, or personal digital assistant). Further details regarding such pump devices can be found in U.S. Pat. No. 8,287,495, previously incorporated by reference above. It is to be appreciated that pump 12 may be optionally configured to deliver one or more additional or other medicaments to a patient.

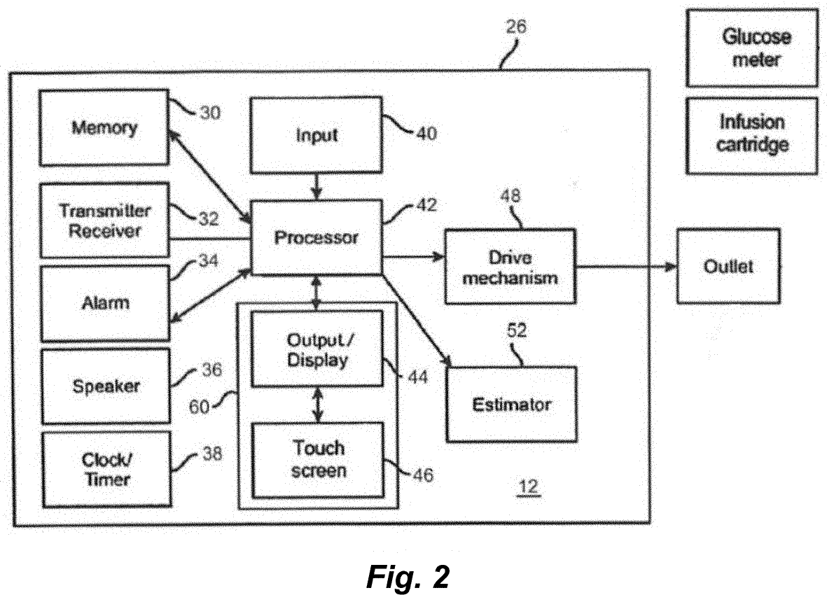

FIG. 2 illustrates a block diagram of some of the features that may be included within the housing 26 of pump 12. The pump 12 can include a processor 42 that controls the overall functions of the pump. The pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices through the transmitter/receiver 32 such as a remote device (e.g., CGM device), a remote control device, or a consumer electronic device (e.g., laptop computer, personal computer, tablet computer, smartphone, electronic watch, electronic health or fitness monitor, or personal digital assistant). In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. The processor 42 may also include programming to receive signals and/or other data from an input device, such as, by way of example, a pressure sensor, a temperature sensor, accelerometer, GPS receiver or the like.

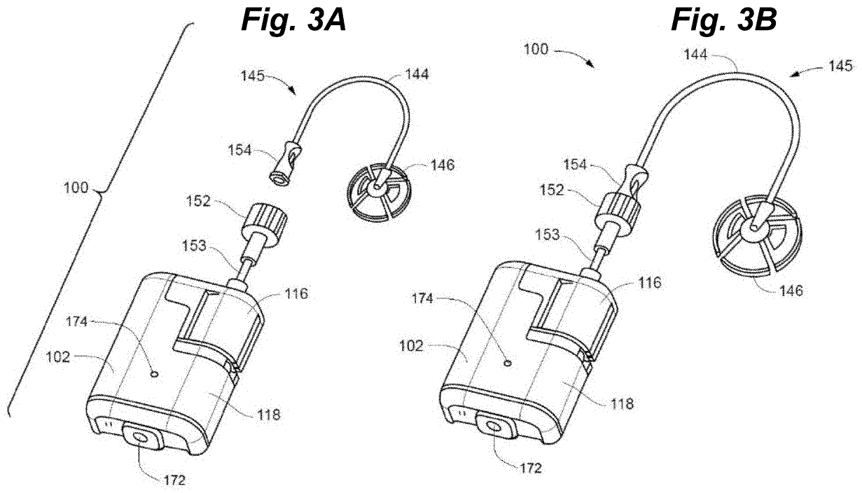

FIGS. 3A-3B depict another infusion pump that can be used in conjunction with one or more embodiments of the ambulatory infusion pump system of the present disclosure. Pump 102 includes a pump drive unit 118 and a medicament cartridge 116. Pump 102 includes a processor that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receive commands from a separate device for control of some or all of the operations of the pump. Such separate device can include, for example, a dedicated remote control device or a consumer electronic device such as a smartphone having a processor executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Pat. No. 10,279,106 and U.S. Patent Publication Nos. 2016/0339172 and 2017/0049957, each of which is hereby incorporated herein by reference in its entirety.

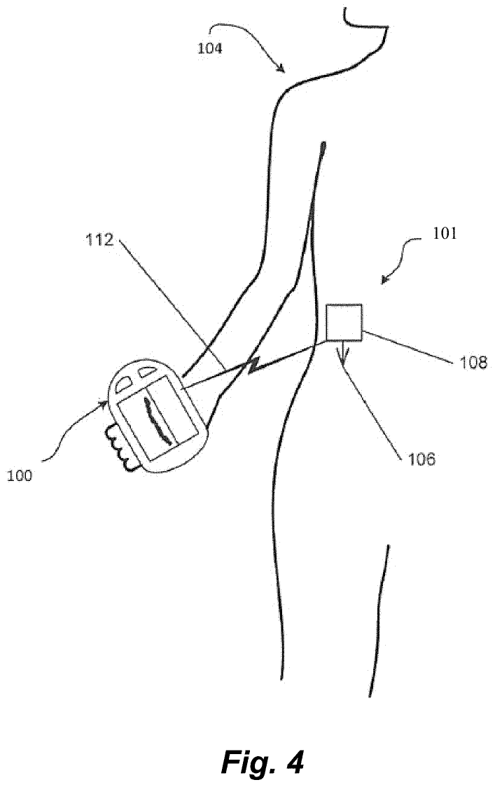

FIG. 4 depicts an example CGM system that can be used in conjunction with one or more embodiments of the ambulatory infusion pump system of the present disclosure. The CGM system includes a sensor 101, a sensor probe 106, a sensor body 108, a receiver, and a monitor (receiver and monitor are depicted as device 100 in FIG. 4). The sensor 101 is removably affixed to a user 104 and includes a sensor probe 106 configured for transcutaneous insertion into the user 104. When placed, the sensor probe 106 reacts with the user's interstitial fluid which produces a signal that can be associated with the user's blood glucose level. The sensor 101 further includes a sensor body 108 that transmits data associated with the signal to the receiver 100 via wired or wireless connection (as represented by arrow line 112). In preferred embodiments, the receiver 100 receives the transmitted data wirelessly by any suitable means of wireless communication. By way of example only, this wireless communication may include a near field communication (NFC) radio frequency (RF) transmitter or a transmitter operating according to a “Wi-Fi” or Bluetooth® protocol, Bluetooth® low energy protocol or the like. Further detail regarding such systems and definitions of related terms can be found in, e.g., U.S. Pat. Nos. 8,311,749, 7,711,402 and 7,497,827, each of which is hereby incorporated by reference in its entirety.

With the infusion pump and CGM interfaced, the CGM can automatically transmit the CGM data to the pump. The pump can then use this data to automatically determine therapy parameters and suggest a therapy adjustment to the user or automatically deliver the therapy adjustment to the user. These therapy parameters including thresholds and target values can be stored in memory located in the pump or, if not located in the pump, stored in a separate location and accessible by the pump processor (e.g., “cloud” storage, a smartphone, a CGM, a dedicated controller, a computer, etc., any of which is accessible via a network connection). The pump processor can periodically and/or continually execute instructions for a checking function that accesses these data in memory, compares them with data received from the CGM and acts accordingly to adjust therapy. In further embodiments, rather than the pump determining the therapy parameters, the parameters can be determined by a separate device and transmitted to the pump for execution. In such embodiments, a separate device such as the CGM or a device in communication with the CGM, such as, for example, a smartphone, dedicated controller, electronic tablet, computer, etc. can include a processor programmed to calculate therapy parameters based on the CGM data that then instruct the pump to provide therapy according to the calculated parameters.

For example, if the CGM readings indicate that the user has or is predicted to have a high blood glucose level, the ambulatory infusion system can automatically calculate an insulin dose sufficient to reduce the user's blood glucose level below a threshold level or to a target level and automatically deliver the dose. Alternatively, the ambulatory infusion system can automatically suggest a change in therapy upon receiving the CGM readings such as an increased insulin basal rate or delivery of a bolus, but can require the user to accept the suggested change prior to delivery rather than automatically delivering the therapy adjustments.

By way of further example, if the CGM readings indicate that the user has or is predicted to have a low blood glucose level (hypoglycemia), the ambulatory infusion system can, for example, automatically reduce or suspend a basal rate, suggest to the user to reduce a basal rate, automatically deliver or suggest that the user initiate the delivery of an amount of a substance such as, e.g., a hormone (glucagon) to raise the concentration of glucose in the blood, automatically suggest that the patient address the hypoglycemic condition as necessary (e.g., ingest carbohydrates), singly or in any desired combination or sequence.

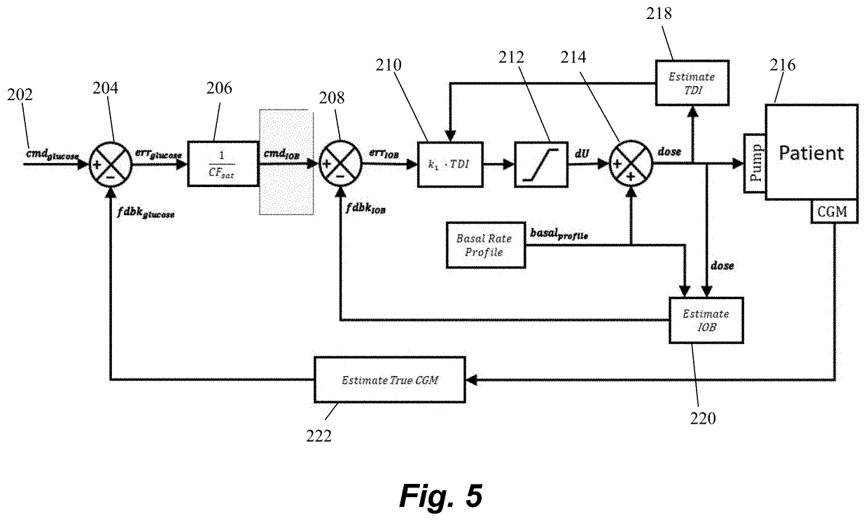

A schematic representation of a control algorithm for automatically adjusting insulin delivery based on CGM data is depicted in FIG. 5. This figure depicts an algorithm for increasing basal rate that utilizes a cascaded loop. The logic for decreasing basal rate is not depicted. In the depicted embodiment, there is a glucose set-point/command (cmd) that is determined at step 202. The glucose set point is a target value at which the algorithm attempts to maintain a user's blood glucose. This value can vary based on a number of factors, including the user's physiology, whether the user is awake or asleep, how long the user has been awake, etc. The glucose set point is compared to the actual CGM feedback (fdbk) at step 204 to determine a glucose error value (err) that is the difference between the set point and the feedback. In various embodiments, the CGM feedback can be a current glucose level reading received from a CGM or can be a predicted future glucose value based on previous glucose readings. For example, the system may predict a glucose level 30 minutes in the future (Gpred30) and utilized the predicted value as the fdbk glucose value. The errGLUCOSE value at step 206 is multiplied by a constant (1/CF), in which CF is the user's correction factor, or amount by which one unit of insulin lowers the user's blood glucose. This calculation determines how much insulin is needed to correct the glucose error, which is how much insulin on board (IOB) is needed in the user's body. This IOB value then determines an appropriate estimated insulin on board (IOB) set point for the patient.

The estimated IOB level determined at step 206 is then taken as the command (cmdIOB) for the inner loop and based on a difference of an IOB feedback value (fdbkIOB) and the cmdIOB set point at step 208, an IOB error value (errIOB) is determined. At step 210, the errIOB value is multiplied by a constant k1 (relating to insulin-dependent glucose uptake in the body) and an estimate of the total daily insulin (TDI) of the user. This adjusts the errIOB to be proportional to the constant and the user's total daily intake of insulin. At step 212, a limiter function is applied to the value calculated at step 210. The limiter function can prevent the calculated amount from being larger or smaller than preset limits. The result is an insulin amount dU, which is the amount by which the user's stored basal rate should be modified. The insulin delivery rate for the user for the next closed loop interval is therefore calculated by modifying the user's stored basal rate profile by the dU value at step 214.

After the dose is calculated, it can be delivered to the user at step 216 and can also be used to update the estimated TDI for the user at step 218. The dose can also be used to update the estimated IOB level for the user at step 220 by comparing the actual insulin delivered to the programmed basal rate. The updated estimated IOB then becomes the new fdbkIOB for the IOB comparison at step 208. When new CGM values are received from the CGM, an estimated true CGM can be determined based on various factors such as, for example, the calibration status of the CGM sensor. The estimated true CGM value then becomes the new fdbkGLUCOSE value for the outer loop comparison with cmdGLUCOSE at step 204 or the estimated true CGM value can be used to update the predicted future glucose level (i.e., Gpred30) for the comparision. The algorithm then proceeds through to calculate a new estimated IOB and to the inner IOB loop for calculation of an insulin dose as described above. In one embodiment, a new CGM value is received every 5 minutes and therefore the algorithm executes as set forth above every 5 minutes.

In addition to automatically modifying basal delivery of insulin based on CGM data as described above, automated insulin delivery systems disclosed herein can also automatically deliver boluses of insulin in certain circumstances. For example, automatic correction boluses can be delivered in situations where a greater amount of insulin is needed more urgently that would be delivered with basal delivery adjustments occurring every 5 minutes. For example, basal insulin may be increased when the user's current or predicted glucose level is above a target glucose level, but if the user's glucose is above a high glucose threshold, such as, for example, 180 mg/dL an automatic correction bolus can be delivered. In embodiments, to mitigate risk of hypoglycemia from automatic correction boluses, auto-boluses can be limited by frequency and/or amount (i.e., delivered in reduced amounts relative to a full correction bolus). In one embodiment, automatic correction boluses can be given only once per hour and are delivered at 60% of a full correction bolus calculated to bring the user's glucose level to the target level.

As noted above, closed loop insulin delivery systems attempt to maintain a user's glucose levels within a predefined range during normal activity, such as, for example, between 112.5 mg/dL and 160 mg/dL. Such systems can also include additional modes, such as Sleep Mode and Exercise Mode, which can provide modified ranges for activities such as sleep and exercise because of the differing bodily requirements during those specific activities. However, some users may benefit from a modified normal activity mode that has a lower and narrower target range for normal activity (i.e., not a specific activity such as exercise or sleep). For example, users that frequently program inaccurate meal boluses and/or forget to bolus, users who frequently spend time above a high threshold such as 180 mg/dL and other users who already control their glucose levels well but desire to spend greater time within the defined glucose range may benefit from a lower and/or narrower target range. In such circumstances, glycemic outcomes can be improved by providing more aggressive control.

In embodiments, more aggressive control can be provided by a user-selectable Alternative Normal Mode feature that is designed to decrease hyperglycemia without significantly increasing the risk of hypoglycemia. A user may be able to select on a user interface of, e.g., the user's pump, dedicated remote control, Smartphone configured for pump control, etc. between a Normal Mode and an Alternative Normal Mode. Both modes can be configured to be employed by the algorithm during normal activity (i.e., not during sleep, exercise, etc.), but can employ different ranges for glycemic control. For example, Normal Mode may employ a standard glucose range between which the closed loop algorithm attempts to maintain the user's glucose levels such as 112.5 mg/dL to 160 mg/dL and Alternative Normal Mode may employ a lower and narrower range such as 90 mg/dL to 130 mg/dL. In other embodiments, the lower end of the range can be anywhere between 70 mg/dL and 90 mg/dL.

Some features of the closed loop algorithm may not be modified in Alternative Normal Mode. For example, a low glucose suspend feature that automatically suspends basal delivery of insulin when a low threshold, such as, e.g., 70 mg/dL, is reached can be the same in both Normal Mode and Alternative Normal Mode. Similarly, an auto-bolus feature that automatically delivers a correction bolus when the user's glucose level exceeds a high threshold, such as, e.g., 180 mg/dL, can be the same in both Normal Mode and Alternative Normal Mode.

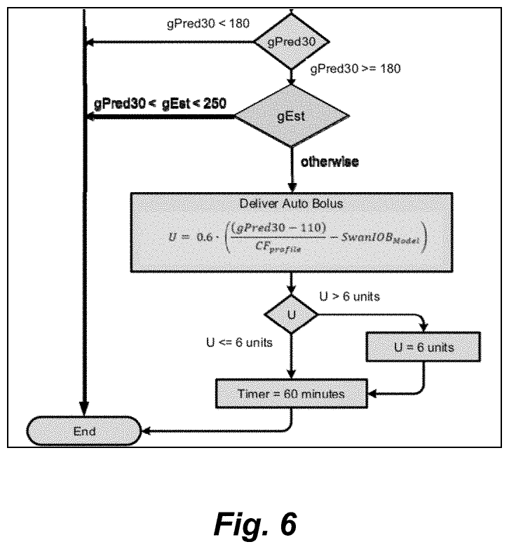

Alternative Normal Mode can also employ a lockout feature that is activated when the system determines that the delivery of an automatic correction bolus would cause a risk of a low glucose level in the user. The lockout feature can disable automatic correction boluses that would otherwise be delivered due to the user's glucose level exceeding the high threshold if, for example, glucose is high, but is falling rapidly. For example, if the user's predicted glucose level in 30 minutes, i.e., gPred30, is less than the user's current estimated glucose level and the user's glucose level is below a certain, higher threshold, such as, e.g., 250 mg/dL, the system can be prohibited from delivering an automatic correction bolus that would normally be delivered due to the user's glucose level being over, e.g., 180 mg/dL. FIG. 6 depicts a schematic representation of a modification to an auto-bolus feature to incorporate such a feature. As can be seen in the Figure, after determining that gPred30 is over 180 mg/dL, but before determining an amount for and delivering an automatic correction bolus, the algorithm incorporates an additional step of determining if gPred30<gEst<250 mg/dL. If so, the automatic bolus is not delivered for that interval. The system can alternatively or additionally prevent delivery of automatic correction boluses based on a risk of a low glucose level determined based on a rate of change of the user's glucose levels falling at more than a predetermined rate and/or if a low glucose alert indicating that a glucose level of a user is below a low threshold had been issued within a predetermined period of time. Further details regarding such an automatic bolus lockout feature can be found in U.S. patent application Ser. No. 17/587,468, which is hereby incorporated by reference herein.

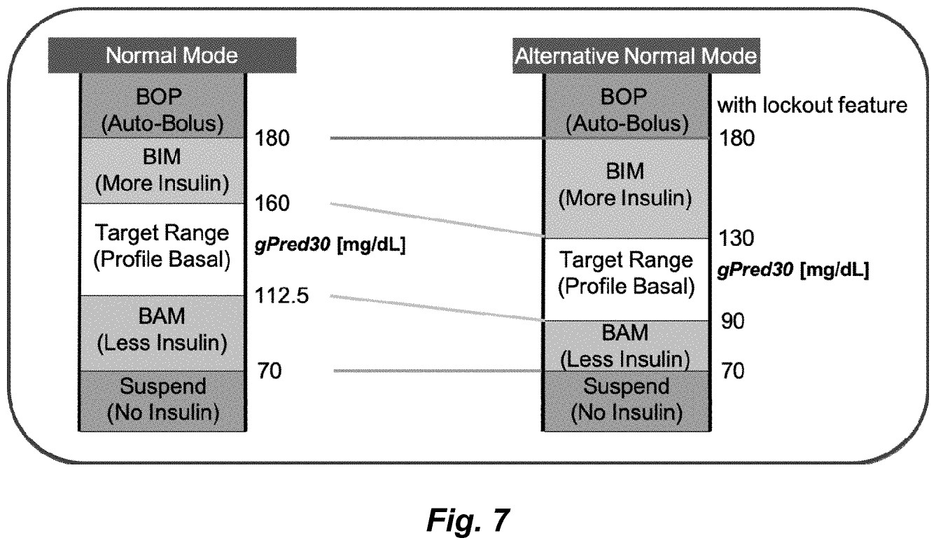

FIG. 7 is a schematic representation of Alternative Normal Mode with respect to Normal Mode. In this embodiment, the low suspend level and the automatic correction bolus threshold remain the same for both Normal Mode and Alternative Normal Mode. In Alternative Normal Mode, an auto-bolus lockout feature as described above can be activated. The target range in which insulin is delivered according to the user's stored basal profile can be narrowed and lowered from 112.5 mg/dL to 160 mg/dL in Normal Mode to 90 mg/dL to 130 mg/dL in Alternative Normal Mode. As such, the range where insulin delivery is increased is widened from 160 mg/dL to 180 mg/dL in Normal Mode to 130 mg/dL to 180 mg/dL in Alternative Normal Mode and the range in which insulin delivery is decreased is narrowed from 70 mg/dL to 112.5 mg/dL in Normal Mode to 70 mg/dL to 90 mg/dL in Alternative Normal Mode. In embodiments where the lower end of the range in Alternate Normal Mode is 70 mg/dL or otherwise equal to the low suspend level, insulin delivery would never be decreased in Alternate Normal Mode, other than to completely suspend delivery based on the low suspend level. Thus, it can be seen that in order to maintain the user's glucose at lower levels in Alternate Normal Mode, insulin delivery is increased at greater frequency and at more glucose levels and decreased less often and at fewer glucose levels than in the traditional Normal Mode.

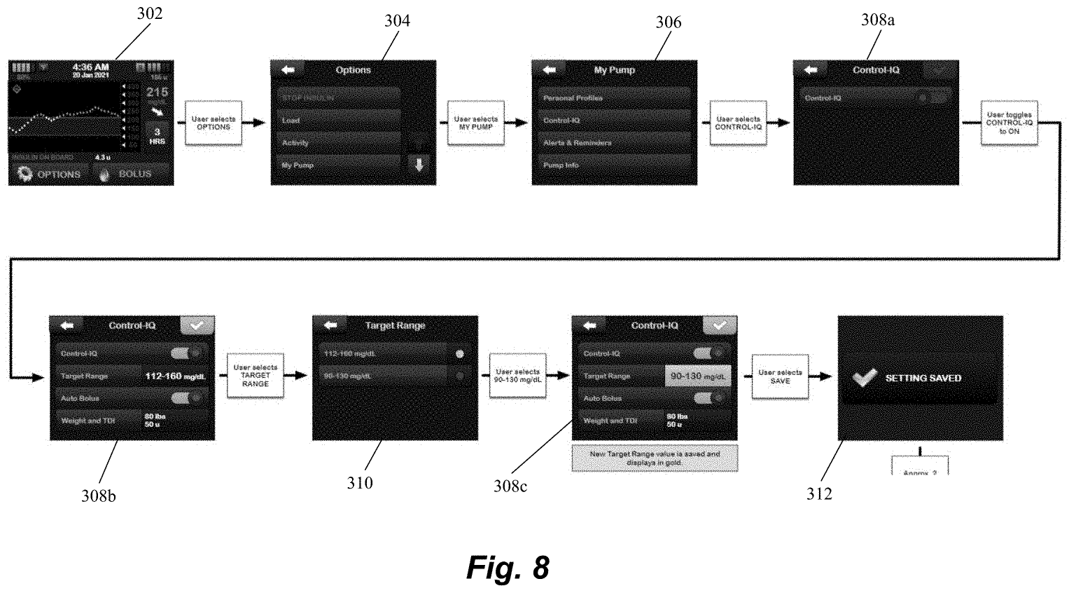

FIG. 8 depicts an exemplary embodiment of a series of user interface screens for selecting alternative delivery modes for closed-loop diabetes therapy according to an embodiment. From a home screen 302, a user can select an OPTIONS object to display an Options menu 304. On the Options menu 304, the user can select a My Pump menu item to display a My Pump menu 306 that, among other menu items, can include menu item relating to a closed loop algorithm used by the device (named “Control IQ” in the depicted embodiment). If the closed loop algorithm is not turned on, the user can first be presented with an option to turn the algorithm on such as that shown on algorithm screen 308a. If the user turns the algorithm on, or if the algorithm is already active when the algorithm menu item is selected in the My Pump menu 306, additional menu items can be displayed as shown on algorithm screen 308b. In a system that includes alternate normal modes having different target ranges such as described herein, a Target Range menu item can be displayed on the algorithm screen 308b. If the Target Range menu item is selected, a Target Range menu 310 can be displayed having different target ranges that are selectable by the user. If the user selects a different target range, the algorithm screen 308c can again be presented with the new target range displayed and the user can be presented with an option to confirm and save the new range. If the user saves the updated setting, a settings confirmation screen 312 can be displayed informing the user of the changed settings. As noted above, selection of a different target range may also automatically change other aspects of the closed loop algorithm (e.g., activation of a bolus lockout feature restricting automatic correction boluses), although the user may not be expressly informed of such modifications. As noted above, these screens represent one exemplary embodiment of a user changing target range and/or normal modes for a closed loop algorithm, and such changes could be done in various other ways.

Simulation analysis has shown that Alternative Normal Mode improved glycemic outcomes compared to Normal Mode across meal and bolus conditions by reducing mean glucose levels by 10 mg/dL, increasing time in range by 3% and decreasing time above 180 mg/dL by 3.9% and time above 250 mg/dL by 1.2%. Further, any hypoglycemic risk with the lower control ranges provided by Alternative Normal Mode is negligible when compared to Normal Mode as it was found that approximately 5 hours after users forget to bolus or over-estimate a bolus size for a large meal there was an increase of only 0.2% for time below 54 mg/dL and 0.9% for time below 70 mg/dL. It has therefore been shown that providing such an alternate mode with lower and/or narrower glucose ranges provides beneficial outcomes for at least some patients.

In embodiments, an ambulatory infusion pump system can include a pump mechanism configured to facilitate delivery of insulin to a user, a user interface, a communications interface adapted to receive glucose levels from a continuous glucose monitor and at least one processor. The at least one processor can be configured to present on the user interface at least two options for calculating and delivering insulin doses according to a closed loop delivery algorithm during normal activity of the user. The at least two options include a normal activity mode having a first glucose range between which the closed loop delivery algorithm attempts to maintain the user's glucose levels and an alternate normal activity mode having a second glucose range. The processor can receive input through the user interface selecting either the normal mode or the alternative normal activity mode, automatically calculate insulin doses with a closed loop delivery algorithm based on glucose levels received from the continuous glucose monitor according to the selected mode, and automatically deliver the insulin doses calculated by the closed loop delivery algorithm to the user with the pump mechanism. If the user has selected the alternate normal activity mode, a bolus lockout feature can further be activated that selectively prevents delivery of automatic correction boluses that would otherwise be delivered to the user's glucose level being over a high threshold.

In some embodiments, the second glucose range is lower and narrower than the first glucose range.

In some embodiments, both a low glucose level and a high glucose level of the second glucose range are lower than a corresponding low glucose level and high glucose level of the first glucose range.

In some embodiments, the at least one processor is further configured to automatically suspend insulin delivery if the user's glucose level is below a low glucose threshold.

In some embodiments, the low glucose threshold is below both the first glucose range and the second glucose range.

In some embodiments, the low glucose threshold is equal to a low glucose level of the second glucose range.

In some embodiments, the at least one processor is configured prevent delivery of automatic correction boluses that would otherwise be delivered due to the user's glucose level being over a high threshold in the alternate normal activity mode with the bolus lockout feature when delivery of an automatic correction bolus would cause a risk of a low glucose level in the user.

In some embodiments, the at least one processor is configured to determine that delivery of an automatic correction bolus would cause a risk of a low glucose level if a rate of change of the user's glucose levels is falling at more than a predetermined rate.

In some embodiments, the at least one processor is configured to determine that delivery of an automatic correction bolus would cause a risk of a low glucose level in the user if a current glucose level of the user is greater than a predicted future glucose level of the user.

In some embodiments, the at least one processor is configured to determine that delivery of an automatic correction bolus would cause a risk of a low glucose level in the user if a low glucose alert indicating that a glucose level of a user is below a low threshold had been issued within a predetermined period of time.

In embodiments, an ambulatory infusion pump system an include a pump mechanism configured to facilitate delivery of insulin to a user, a user interface, a communications interface adapted to receive glucose levels from a continuous glucose monitor and at least one processor. The at least one processor can be configured to present on the user interface at least two options for calculating and delivering insulin doses according to a closed loop delivery algorithm during normal activity of the user. The at least two options include a normal activity mode having a first glucose range between which the closed loop delivery algorithm attempts to maintain the user's glucose levels and an alternate normal activity mode having a second glucose range that is lower and narrower than the first glucose range. The processor can receive input through the user interface selecting either the normal mode or the alternative normal activity mode, automatically calculate insulin doses with a closed loop delivery algorithm based on glucose levels received from the continuous glucose monitor according to the selected mode, and automatically deliver the insulin doses calculated by the closed loop delivery algorithm to the user with the pump mechanism.

In some embodiments, the at least one processor is configured to selectively prevent delivery of automatic correction boluses that would otherwise be delivered due to the user's glucose level being over a high threshold in the alternate normal activity mode with a bolus lockout feature.

In some embodiments, the at least one processor is configured prevent delivery of automatic correction boluses that would otherwise be delivered due to the user's glucose level being over a high threshold in the alternate normal activity mode with the bolus lockout feature when delivery of an automatic correction bolus would cause a risk of a low glucose level in the user.

In some embodiments, the at least one processor is configured to determine that delivery of an automatic correction bolus would cause a risk of a low glucose level if a rate of change of the user's glucose levels is falling at more than a predetermined rate.

In some embodiments, the at least one processor is configured to determine that delivery of an automatic correction bolus would cause a risk of a low glucose level in the user if a current glucose level of the user is greater than a predicted future glucose level of the user.

In some embodiments, the at least one processor is configured to determine that delivery of an automatic correction bolus would cause a risk of a low glucose level in the user if a low glucose alert indicating that a glucose level of a user is below a low threshold had been issued within a predetermined period of time.

In some embodiments, both a low glucose level and a high glucose level of the second glucose range are lower than a corresponding low glucose level and high glucose level of the first glucose range.

In some embodiments, the at least one processor is further configured to automatically suspend insulin delivery if the user's glucose level is below a low glucose threshold.

In some embodiments, the low glucose threshold is below both the first glucose range and the second glucose range.

In some embodiments, the low glucose threshold is equal to a low glucose level of the second glucose range.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10,541,987; 10,569,016; 10,736,037; 10,888,655; 10,994,077; 11,116,901; and 11,224,693 and commonly owned U.S. Patent Publication Nos. 2009/0287180; 2012/0123230; 2013/0053816; 2014/0276423; 2014/0276569; 2014/0276570; 2018/0071454; 2019/0240398; 2019/0307952; 2020/0206420; 2020/0261649; 2020/0306445; 2020/0329433; 2020/0368430; 2020/0372995; 2021/0001044; 2021/0113766; 2021/0154405; and 2021/0353857 and commonly owned U.S. patent application Ser. No. 17/368,968; Ser. No. 17/459,129; Ser. No. 17/517,885 and Ser. No. 17/573,705.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An ambulatory infusion pump system, comprising:
a pump mechanism configured to facilitate delivery of insulin to a user;
a user interface;
a communications interface adapted to receive glucose levels from a continuous glucose monitor; and
at least one processor configured to:
present on the user interface at least two options for calculating and delivering insulin doses according to a closed loop delivery algorithm during normal activity of the user, including a normal activity mode having a first glucose range between which the closed loop delivery algorithm attempts to maintain the user's glucose levels and an alternate normal activity mode having a second glucose range;
receive input through the user interface selecting the alternative normal activity mode;
automatically calculate insulin doses with a closed loop delivery algorithm based on glucose levels received from the continuous glucose monitor according to the selected option;
automatically deliver the insulin doses calculated by the closed loop delivery algorithm to the user with the pump mechanism; and
selectively prevent delivery of automatic correction boluses that would otherwise be delivered due to the user's glucose level being over a high threshold in the alternate normal activity mode with a bolus lockout feature.

2. The ambulatory infusion pump system of claim 1, wherein the second glucose range is lower and narrower than the first glucose range.

3. The ambulatory infusion pump system of claim 2, wherein both a low glucose level and a high glucose level of the second glucose range are lower than a corresponding low glucose level and high glucose level of the first glucose range.

4. The ambulatory infusion pump system of claim 1, wherein the at least one processor is further configured to automatically suspend insulin delivery if the user's glucose level is below a low glucose threshold.

5. The ambulatory infusion pump system of claim 4, wherein the low glucose threshold is below both the first glucose range and the second glucose range.

6. The ambulatory infusion pump system of claim 4, wherein the low glucose threshold is equal to a low glucose level of the second glucose range.

7. The ambulatory infusion pump system of claim 1, wherein the at least one processor is configured prevent delivery of automatic correction boluses that would otherwise be delivered due to the user's glucose level being over a high threshold in the alternate normal activity mode with the bolus lockout feature when delivery of an automatic correction bolus would cause a risk of a low glucose level in the user.

8. The ambulatory infusion pump system of claim 7, wherein the at least one processor is configured to determine that delivery of an automatic correction bolus would cause a risk of a low glucose level if a rate of change of the user's glucose levels is falling at more than a predetermined rate.

9. The ambulatory infusion pump system of claim 7, wherein the at least one processor is configured to determine that delivery of an automatic correction bolus would cause a risk of a low glucose level in the user if a current glucose level of the user is greater than a predicted future glucose level of the user.

10. The ambulatory infusion pump system of claim 7, wherein the at least one processor is configured to determine that delivery of an automatic correction bolus would cause a risk of a low glucose level in the user if a low glucose alert indicating that a glucose level of a user is below a low threshold had been issued within a predetermined period of time.

11. An ambulatory infusion pump system, comprising:
a pump mechanism configured to facilitate delivery of insulin to a user;
a user interface;
a communications interface adapted to receive glucose levels from a continuous glucose monitor; and
at least one processor configured to:
present on the user interface at least two options for calculating and delivering insulin doses according to a closed loop delivery algorithm during normal activity of the user, including a normal activity mode having a first glucose range between which the closed loop delivery algorithm attempts to maintain the user's glucose levels and an alternate normal activity mode having a second glucose range that is lower and narrower than the first glucose range;
receive input through the user interface selecting the alternative normal activity mode;
automatically calculate insulin doses with a closed loop delivery algorithm based on glucose levels received from the continuous glucose monitor according to the selected option; and
automatically deliver the insulin doses calculated by the closed loop delivery algorithm to the user with the pump mechanism.

12. The ambulatory infusion pump of claim 11, wherein the at least one processor is configured to selectively prevent delivery of automatic correction boluses that would otherwise be delivered due to the user's glucose level being over a high threshold in the alternate normal activity mode with a bolus lockout feature.

13. The ambulatory infusion pump system of claim 12, wherein the at least one processor is configured prevent delivery of automatic correction boluses that would otherwise be delivered due to the user's glucose level being over a high threshold in the alternate normal activity mode with the bolus lockout feature when delivery of an automatic correction bolus would cause a risk of a low glucose level in the user.

14. The ambulatory infusion pump system of claim 13, wherein the at least one processor is configured to determine that delivery of an automatic correction bolus would cause a risk of a low glucose level if a rate of change of the user's glucose levels is falling at more than a predetermined rate.

15. The ambulatory infusion pump system of claim 14, wherein the at least one processor is configured to determine that delivery of an automatic correction bolus would cause a risk of a low glucose level in the user if a current glucose level of the user is greater than a predicted future glucose level of the user.

16. The ambulatory infusion pump system of claim 13, wherein the at least one processor is configured to determine that delivery of an automatic correction bolus would cause a risk of a low glucose level in the user if a low glucose alert indicating that a glucose level of a user is below a low threshold had been issued within a predetermined period of time.

17. The ambulatory infusion pump system of claim 11, wherein both a low glucose level and a high glucose level of the second glucose range are lower than a corresponding low glucose level and high glucose level of the first glucose range.

18. The ambulatory infusion pump system of claim 11, wherein the at least one processor is further configured to automatically suspend insulin delivery if the user's glucose level is below a low glucose threshold.

19. The ambulatory infusion pump system of claim 18, wherein the low glucose threshold is below both the first glucose range and the second glucose range.

20. The ambulatory infusion pump system of claim 18, wherein the low glucose threshold is equal to a low glucose level of the second glucose range.
